(19) United States
(12) Patent Application Publication
(10) Pub. No.: US 2004/0014102 A1
(43) Pub. Date: Jan. 22, 2004

(54) High density parallel printing of microarrays

(76) Inventors: Shiping Chen, Fremont, CA (US); Zhiyong Shen, Fremont, CA (US); Yuling Luo, Castro Valley, CA (US)

Correspondence Address: MORRISON & FOERSTER LLP, 755 PAGE MILL RD, PALO ALTO, CA, 94304-1018, US

(21) Appl. No.: 10/431,169

(22) Filed: May 6, 2003

Related U.S. Application Data
(63) Continuation-in-part of application No. 09/791,994, filed on Feb. 22, 2001, now Pat. No. 6,594,432.
(63) Continuation-in-part of application No. 09/791,998, filed on Feb. 22, 2001.
(60) Provisional application No. 60/183,737, filed on Feb. 22, 2000.
(60) Provisional application No. 60/188,872, filed on Mar. 13, 2000.
(60) Provisional application No. 60/216,265, filed on Jul. 6, 2000.
(60) Provisional application No. 60/220,085, filed on Jul. 21, 2000.
(60) Provisional application No. 60/244,711, filed on Oct. 30, 2000.
(60) Provisional application No. 60/183,737, filed on Feb. 22, 2000.
(60) Provisional application No. 60/188,872, filed on Mar. 13, 2000.
(60) Provisional application No. 60/216,265, filed on Jul. 6, 2000.
(60) Provisional application No. 60/220,085, filed on Jul. 21, 2000.
(60) Provisional application No. 60/244,413, filed on Oct. 30, 2000.
(60) Provisional application No. 60/378,485, filed on May 6, 2002.
(60) Provisional application No. 60/401,485, filed on Aug. 5, 2002.

(51) Int. Cl. C12Q 1/68; B05D 3/00; C12M 1/34

(52) U.S. Cl. 435/6; 427/2.11; 435/287.2

(57) ABSTRACT

A method for printing a microarray on a substrate having a substrate surface is provided. At least one probe reservoir and at least one capillary bundle comprising a plurality of individual capillaries is provided. The output ends of the individual capillaries are secured in a print head such that the output ends of the capillaries are substantially coplanar in an array in a facet of the print head. The capillaries have a capillary pitch P. Probe is transported from at least one probe reservoir to the output ends of the capillaries. An array of probes are printed on the substrate such that the printed probes have a probe pitch of approximately P/N where N is an integer greater than one. Also provided is a method of associating proximal and distal ends of a plurality of capillaries in a capillary bundle. A plurality of fluids are loaded into the distal ends of the plurality of capillaries, each capillary having a unique fluid being loaded therein. The plurality of fluids are transported from the distal ends of the plurality of capillaries to the proximal ends and printed onto a substrate to form an array of spots, each spot corresponding to one of the plurality of fluids. One of the capillaries is registered by identifying the fluid forming one of the spots in the array of spots, matching the identified fluid with one of the plurality of fluids loaded into the distal ends of the capillaries, and correlating the location of the spot with the capillary loaded with the matched fluid.

### CROSS-REFERENCE TO RELATED APPLICATIONS

This is a continuation-in-part of U.S. application Ser. No. 09/791,994, filed Feb. 22, 2001, which claims the benefit of U.S. Provisional Application Nos.: 60/183,737, filed on Feb. 22, 2000; 60/188,872, filed on Mar. 13, 2000; 60/216,265, filed on Jul. 6, 2000; 60/220,085, filed on Jul. 21, 2000; 60/244,711, filed on Oct. 30, 2000. This is also a continuation-in-part of U.S. application Ser. No. 09/791,998, filed Feb. 22, 2001, which claims the benefit of U.S. Provisional Application Nos.: 60/183,737, filed on Feb. 22, 2000; 60/188,872, filed on Mar. 13, 2000; 60/216,265, filed on Jul. 6, 2000; 60/220,085, filed on Jul. 21, 2000; 60/244,413, filed on Oct. 30, 2000. This also claims the benefit of U.S. Provisional Application Nos. 60/378,485, filed on May 6, 2002, and 60/401,485, filed on Aug. 5, 2002. All of the above applications are incorporated by reference herein in their entireties as if fully set forth below.

### BACKGROUND OF THE INVENTION

A microarray is an array of spots of biological or chemical samples (“probes”) immobilized at predefined positions on a substrate. Each spot contains a number of molecules of a biological or chemical material. To interrogate the array, the microarray is flooded with a fluid containing one or more biological or chemical samples (the “target”), elements of which typically interact with one or more complementary probes on the microarray. In DNA microarrays in particular, the probes are oligonucleotide or cDNA strains, and the target is a fluorescent or radioactive-labeled DNA sample. The molecular strands in the target hybridize with complementary strands in the probe microarray. The hybridized microarray is inspected by a microarray reader, which detects the presence of the radioactive labels or which stimulates the fluorescent labels to emit light through excitation with a laser or other energy sources. The reader detects the position and strength of the label emission in the microarray. Since the probes are placed in predetermined and thus known positions in the microarray, the presence and quantity of target sequences in the fluid are identified by the position at which fluorescence or radiation is detected and the strength of the fluorescence or radiation.

Microarray technology can provide an extremely useful tool to conduct biological or chemical experiments in a massively parallel fashion because of the large number of different probes that can be fabricated onto the microarray. It can be particularly powerful in screening, profiling, and identifying DNA samples.

Microarrays may be provided as two-dimensional probe matrices fabricated on solid glass or nylon substrates. Because the target samples are generally difficult and/or expensive to produce, it is highly desirable to perform assays on as many features as possible on a single microarray. This calls for a significant increase in probe density and quantity on a single substrate. In general, microarrays with probe pitch smaller than 500 μm (i.e., density larger than 400 probes per square centimeter) is referred as high density microarrays, otherwise, they are “low density” microarrays.

Photolithographic and robotic spotting techniques have been used to fabricate microarrays. The photolithographic technique adapts the same fabrication process for electronic integrated circuits to synthesize probes in situ base by base. This technique typically requires a large capital outlay for equipment running up to hundreds of millions of dollars. The initial setup of new microarray designs can be also very expensive due to the high cost of producing photo masks. This technique is therefore only viable in mass production of standard microarrays at a very high volume. Even at high volumes, the complexity in synthesis can still limit the production throughput, resulting in a high microarray cost. This complexity can also limit the length of the synthesized DNA strain to the level of a short oligonucleotide (˜25 bases), which reduces the specificity and sensitivity of hybridization in some applications.

A robotic spotting technique uses a specially designed mechanical robot, which produces a probe spot on the microarray by dipping a pin head into a fluid containing an off-line synthesized DNA and then spotting it onto the slide at a pre-determined position. The pins are washed and dried prior to the spotting of each different probe in the microarray. In current designs of such robotic systems, the spotting pin and/or the stage carrying the microarray substrates move along the XYZ axes in coordination to deposit samples at controlled positions of the substrates. Because a microarray contains a very large number of different probes, this technique, although highly flexible, is inherently very slow. Even though the speed can be enhanced by employing multiple pin-heads and spotting multiple slides before washing, production throughput remains very low. This technique is therefore not suitable for high volume mass production of microarrays.

In addition to the established quill-pin spotting technologies, there are a number of microarray fabrication techniques that are being developed. These include the inkjet technology and capillary spotting.

Inkjet technology has been deployed to deposit either cDNA/oligonucleotides or individual nucleotides at defined positions on a substrate to produce an oligonucleotide microarray through in situ synthesis. Consequently, an oligonucleotide is produced in situ one base at a time by delivering monomer-containing solutions onto selected locations, reacting the monomer, rinsing the substrate to remove excess monomers, and drying the substrate to prepare it for the next spot of monomer reactant;

An emerging spotting technique uses capillaries instead of pins to spot DNA probes onto the support. Four references discuss capillary-based spotting techniques for array fabrication: WO 98/29736, “Multiplexed molecular analysis apparatus and method”, by Genometrix Inc.; WO 00/01859, “Gene pen devices for array printing”, by Orchid Biocomputer Inc.; WO 00/13796, “Capillary printing system”, by Incyte Pharmaceuticals Inc.; and WO 99/55461, “Redrawn capillary imaging reservoir”, by Corning Inc.

In summary, due to the high cost of production, microarrays fabricated with existing technologies can be extremely expensive and impractical, particularly as a single use lab supply.

### SUMMARY OF INVENTION

In accordance with aspects of the present invention, there is provided a method for printing a microarray on a substrate having a substrate surface. At least one probe reservoir is provided. Furthermore, at least one capillary bundle comprising a plurality of individual capillaries is provided. Each of the capillaries has an input end and an output end. The output ends of the individual capillaries are secured in a print head such that the output ends of the capillaries are substantially coplanar in an array in a facet of the print head. The capillaries have a capillary pitch P. The input ends of the individual capillaries are placed in fluid communication with at least one probe reservoir. Probe is transported from at least one probe reservoir to the output ends of the capillaries. An array of probes are printed on the substrate such that the printed probes have a probe pitch of approximately P/N where N is an integer greater than one.

In accordance with another aspect of the invention, there is provided a method for printing a microarray. A substrate for receiving a probe array having a probe pitch p is provided. At least one capillary bundle comprising a plurality of individual capillaries is also provided. Each of the capillaries has an input end and an output end. The output ends of the individual capillaries are secured in a print head such that the output ends of the capillaries are substantially coplanar in an array in a facet of the print head such that the capillaries have a capillary pitch P. The capillary pitch P is an integer multiple of the probe pitch p where the integer is greater than one. The input ends of the individual capillaries are placed in fluid communication with at least one probe reservoir and probe is transported from the at least one probe reservoir to the output ends of the capillaries. An N² number of prints is printed to deposit N² sets of probes onto the substrate to form a probe array having a probe pitch p.

In accordance with another aspect of the invention, there is provided a microarray printing system comprising at least one probe reservoir and a substrate for receiving a probe array having a probe pitch p. The system further includes at least one capillary bundle comprising a plurality of individual capillaries. Each of the capillaries has an input end and an output end. The input ends are in fluid communication with the at least one probe reservoir. The output ends of the individual capillaries are secured in a print head such that the output ends of the capillaries are substantially coplanar in an array in a facet of the print head such that the capillaries have a capillary pitch P. The capillary pitch P is an integer multiple of the probe pitch p where the integer is greater than one. The system is configured to print N² number of prints depositing N² number of sets of probes onto the substrate to form a probe array having a pitch p.

In accordance with yet another aspect of the invention, there is provided a microarray printing system comprising at least one probe reservoir and a substrate configured to receive a probe array having a probe pitch p. The system includes a plurality of fluid dispensing members each having a distal end and a proximal end. Each fluid dispensing member is in fluid communication with at least one probe reservoir. The proximal ends of the individual fluid dispensing members are secured such that the proximal ends of the dispensing members are substantially coplanar in an array in a facet of the print head such that the capillaries have a pitch P. The printing system is configured for printing a probe array having a probe pitch p wherein P=Np and N is an integer greater than one.

In accordance with another aspect of the invention, there is provided a method for fabricating a microarray substrate. A substrate having a substrate surface is provided. A layer of photo resist is applied to the substrate surface. The layer of photo resist is patterned into an array of probe locations and the layer of photo resist is removed from an area surrounding the probe locations. A hydrophobic layer is applied on the area surrounding the probe locations and the layer of photo resist from the probe locations is removed to expose the substrate surface. The probe locations are functionalized for probe binding.

In accordance with another aspect of the invention, there is provided a microarray printing system comprising at least one reservoir and a substrate configured to receive a probe array. The system includes at least one print head comprising a plurality of fluid dispensing members. Each fluid dispensing member has a distal end and a proximal end. Each fluid dispensing member is in fluid communication with at least one reservoir. The proximal ends of the individual fluid dispensing members are secured such that the proximal ends of the dispensing members are substantially coplanar in an array in a facet of the print head. The printing system is configured to print at least two arrays on the substrate—a first array and a second array. The first array has a pitch p and a pattern. The second array has the same pitch and pattern as the first array. The printing system is configured to print a first array of first material and to print at least a second array of second material onto the first array of first material.

In accordance with another aspect of the invention, there is provided a method for printing a microarray. The method includes the step of providing a substrate having a substrate surface. At least one reservoir is also provided. At least one print head comprising a plurality of fluid dispensing members is provided. Each fluid dispensing member has a distal end and a proximal end. Each fluid dispensing member is in fluid communication with at least one reservoir. The proximal ends of the individual fluid dispensing members are secured such that the proximal ends of the fluid dispensing members are substantially coplanar in an array in a facet of the print head. A first array of first material is printed onto the substrate. And, at least a second array of at least second material is printed onto the first array.

In accordance with another aspect of the invention, there is provided a method for printing a microarray using at least one capillary bundle comprising a plurality of individual capillaries, each of the capillaries having an input end and an output end, wherein the output ends of the individual capillaries are secured in a print head. The method comprises: transporting probe from at least one probe reservoir to the output ends of the capillaries; printing a first array of probes on a substrate; and printing a second array of probes on the substrate, said second array of probes overlapping and offset from the first array of probes such that at least some of the probes in the second array of probes are located between the probes in the first array of probes.

In some embodiments, the method may further comprise: after printing the first array of probes but before printing the second array of probes, translating the print head a distance less than a width of the first array of probes. In other embodiments, the method may further comprise: printing the first array of probes on the substrate using a first capillary bundle; and printing the second array of probes on the substrate using a second capillary bundle. In other embodiments, the method may further comprise: providing an array of functionalized patches on the substrate, wherein the first array of probes is printed on a first subset of the array of functionalized patches and the second array of probes is printed on a second subset of the array of functionalized patches. In some embodiments, said providing the array of functionalized patches comprises providing patches that are hydrophilic. In some embodiments, said providing the array of functionalized patches further comprises providing hydrophobic areas surrounding the hydrophilic patches.

In accordance with another aspect of the invention, there is provided a method of associating proximal and distal ends of a plurality of capillaries in a capillary bundle. The method comprises: loading a plurality of fluids into the distal ends of the plurality of capillaries, each capillary having a unique fluid being loaded therein; transporting the plurality of fluids from the distal ends of the plurality of capillaries to the proximal ends; printing the plurality of fluids from the proximal ends of the plurality of capillaries onto a substrate to form an array of spots, each spot corresponding to one of the plurality of fluids; and registering one of the capillaries by identifying the fluid forming one of the spots in the array of spots, matching the identified fluid with one of the plurality of fluids loaded into the distal ends of the capillaries, and correlating the location of the spot with the capillary loaded with the matched fluid.

In some embodiments, said registering one of the capillaries is performed for each of the plurality of capillaries in the capillary bundle. In other embodiments, said loading a plurality of fluids into the distal ends of the plurality of capillaries comprises loading a plurality of colored fluids into the distal ends of the plurality of capillaries, each fluid having a uniquely identifiable color. In some embodiments, said identifying the fluid forming one of the array of spots comprises scanning the array of spots using a microarray scanner to identify the color of the fluid forming one of the array of spots.

In yet other embodiments, said loading a plurality of fluids into the distal ends of the plurality of capillaries comprises loading a plurality of fluids into the distal ends of the plurality of capillaries, each fluid including a unique combination of one or more oligonucleotides, each of the one or more oligonucleotides having a known sequence. The unique combination of one or more oligonucleotides may comprise a unique combination of M oligonucleotides, wherein M is an integer greater than one. The unique combination of M oligonucleotides may further comprise a unique combination of different concentrations of M oligonucleotides. The loading the plurality of fluids into the distal ends of the plurality of capillaries may comprise loading a plurality of fluids into the distal ends of the plurality of capillaries, each fluid including: (1) a reference oligonucleotide having a known sequence; (2) the unique combination of different concentrations of M oligonucleotides; and (3) a compensation oligonucleotide having a known sequence and concentration selected such that a total oligonucleotide concentration in each fluid in the plurality of fluids is approximately equal.

In other embodiments, said printing the plurality of fluids onto the substrate comprises printing the plurality of fluids onto M substrates to form an array of spots onto each of the M substrates; and said identifying the fluid forming one of the spots in the array of spots comprises: hybridizing the array of spots on each of the M substrates with one of a plurality of M target solutions, each one of the M target solutions including labeled targets complimentary to the reference oligonucleotide and labeled targets complimentary to one of the oligonucleotides in the unique combination of M oligonucleotides; and scanning the array of spots on each of the M substrates to identify the sequence and concentration of oligonucleotides in each spot.

Other features and aspects of the invention will become apparent from the following detailed description, taken in conjunction with the accompanying drawings which illustrate, by way of example, the features in accordance with embodiments of the invention. The summary is not intended to limit the scope of the invention, which is defined solely by the claims attached hereto.

### BRIEF DESCRIPTION OF THE DRAWINGS

FIGS. 1A-1D illustrate the fabrication of a substrate surface.

FIG. 2 is a schematic diagram of a microarray fabrication system.

FIGS. 3A-3D illustrate the fabrication of a capillary bundle using a guide plate.

FIG. 4 illustrates a cross-sectional view of a portion of a capillary bundle.

FIG. 5 illustrates a desired probe spot array or a probe patch array for probe binding.

FIGS. 6A-6F illustrate steps in printing a microarray using a differential printing technique in accordance with embodiments of the present invention.

In the following description, reference is made to the accompanying drawings which form a part thereof, and which illustrate several embodiments of the present invention. It is understood that other embodiments may be utilized and structural and operational changes may be made without departing from the scope of the present invention.

### DETAILED DESCRIPTION OF THE INVENTION

In the description below, a DNA microarray is used as one embodiment of the invention. The techniques described herein can also be used to produce microarrays of a wide range of biological and chemical probe materials which include but are not limited to deoxyribonucleic acids (DNA), ribonucleic, acids (RNA), synthetic oligonucleotides, antibodies, cells, tissue, proteins, peptides, lectins, modified polysaccharides, synthetic composite macromolecules, functionalized nanostructures, synthetic polymers, modified/blocked nucleotides/nucleosides, modified/blocked amino acids, fluorophores, chromophores, ligands, chelates, haptens, drug compounds, and chemical compounds that have associated substance which binds, associates, or interacts with the probe material. The samples being deposited on the microarray substrate using the technology disclosed herein can take or be carried by any physical form that can be transported through a capillary. These include but are not limited to aqueous or non-aqueous fluid, gel, paste, bead, powder and particles suspended in aqueous or non-aqueous liquid.

The substrate may be formed of any material on which the probes can be deposited. The substrate itself may be capable of immobilizing the particular probes used, or the substrate may be capable of modification (for example, by coating) so that it is capable of such immobilization. The substrate may be porous or nonporous materials. Exemplary materials for the substrate of the present invention include silica, glass, metals, plastics, and polymers.

For immobilizing polynucleotides and polypeptides, glass may be used as the substrate material because polynucleotides and polypeptides can be covalently attached to a treated glass surface and glass emits minimal fluorescent noise signals. The glass may be layered on another material, or it may be core or base material of the apparatus, or both. Another example of a substrate includes a plastic or polymer tape as a base substrate, with a coating of silica for probe embodiment. In this embodiment, a further layer of metallic material may be added, either on the opposite side of the tape from the silica layer, or sandwiched between the silica layer and the polymer or plastic.

In some embodiments, the microarray substrate is a suitable solid support with a surface that is flat or geometrically matches the shape of the print head. In one variation, an array of functional patches is produced on the surface. The area inside the patch is chemically functionalized so that it is capable of binding biochemical probes to the solid surface. The area outside of the patch is made to be non-binding to the biochemical probes. In addition, the area inside the patch can be made hydrophilic while the area outside is made hydrophobic. The binding between probe and surface can be covalent or noncovalent.

In the case in which probes are attached to the substrate covalently, a variety of approaches to bind an oligonucleotide to the solid substrate are available. By using chemically reactive solid substrates, one may provide for a chemically reactive group to be present on the nucleic acid, which will react with the chemically active solid substrate surface. One may form silicon esters for covalent bonding of the nucleic acid to the surface. Instead of silicon functionalities, one may use organic addition polymers, e.g. styrene, acrylates and methacrylates, vinyl ethers and esters, and the like, where functionalities are present which can react with a functionality present on the nucleic acid. Amino groups, activated halides, carboxyl groups, mercaptan groups, epoxides, and the like, may also be provided in accordance with conventional ways. The linkages may be amides, amidines, amines, esters, ethers, thioethers, dithioethers, and the like. Methods for forming these covalent linkages may be found in U.S. Pat. No. 5,565,324 and references cited therein.

Alternatively, the probes may be attached to the substrate or to beads non-covalently by, e.g., functionalizing the surface of the substrate and the probe to provide binding moieties on each. Generally, this will be accomplished by providing each of the probe and the support with one of a pair of corresponding affinity binding partners, such that the probe and the support may be bound together selectively, and if desired, reversibly. Many techniques for binding oligonucleotide and glass surfaces are well known in the field.

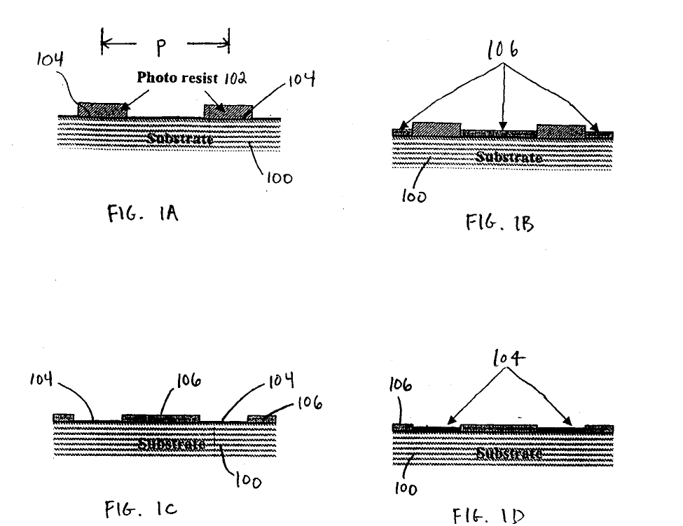

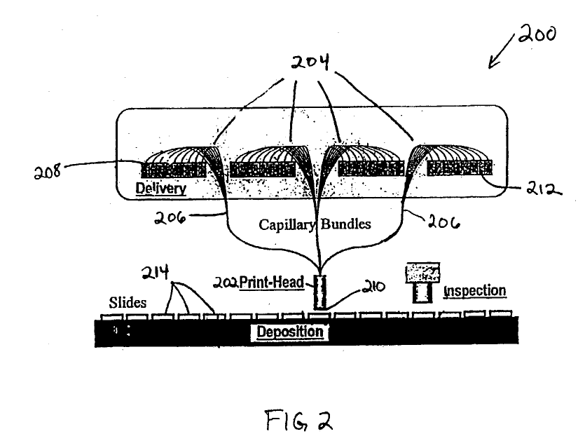

Microfabrication techniques widely used in the semiconductor industry can be employed to produce the substrate surface patch array. Referring now to FIGS. 1A-1D, there is shown one method for fabricating functionalized hydrophilic patches on a glass substrate 100. As shown in FIG. 1A, the first step is to coat the substrate 100 with a layer of photo resist 102. Using lithography techniques well known in the art, the layer of photo resist 102 is then etched to pattern an array of patch areas 104 such that the desired patch locations are protected by the photo resist film 102. The center-to-center distance between patches 104 is defined as the patch pitch “p”. In the next step, a hydrophobic layer 106 is coated on the unprotected area outside of and surrounding the patch areas 104 as shown in FIG. 1B. Then, the photo resist layer 102 is removed to expose the original substrate surface, thereby, forming the patch areas 104 as shown in FIG. 1C. Finally, the exposed substrate surface is functionalized for probe binding using methods well known in the art as shown in FIG. 1D.

A microarray fabrication system 200 is illustrated schematically in FIG. 2. The system 200 includes a print head 202 comprising a plurality of fluid dispensing members such as capillaries 204 bound into at least one capillary bundle 206. Each capillary 204 has two ends, an unbound distal or input end 208 and a bound proximal or output end 210. The input ends 208 of the capillaries 204 are fluidly linked to at least one reservoir 212, such as a microtitre well plate, containing a chemical agent to be assayed. The output ends 210 are bound closely together to form a capillary bundle at the print head 202. The chemical agent is delivered from the reservoir 212 to the capillary input end 208 and through the capillary 204 to the output end 210. From the output end 210, the chemical compound is delivered to a surface of a microarray substrate 214.

A print head can be, for example, a solidified piece of polymer such as a thermo-setting or other polymer (for example, an epoxy polymer) that surrounds the output ends 210 of the capillaries 204, and its facet or face adjacent to the substrate 214 can be fabricated to conform to the surface contour of the microarray substrate 214 in order to facilitate uniformed probe deposition.

The print head 202 can be solid or have sufficient flexibility to conform to the substrate surface on which a microarray is to be printed. The print head 202 may contain a single capillary bundle or, as shown in FIG. 2, multiple capillary bundles 206. In some embodiments of the multiple bundle configuration, the outline shape of each bundle can be rectangular or square so that the capillary bundles can easily be assembled to form a structured matrix in a rectangular print head. In this way, 1) the print head can be configured to print on most or all of the surface area of a standard microscope slide; 2) the position and orientation of each bundle in the system is known; and 3) it is easier to identify each capillary in a bundle. Alternatively, the outline shape of each bundle could be round or in other shapes.

Capillaries used in the system can be made of, for example, silica or other suitable materials such as glass, ceramics, polymer or metal. The capillaries conduct the probes of interest from the input ends of the capillaries to the output ends of the capillaries. Thus, capillaries that are bundled to form a print head may be manufactured from a material that does not remove a substantial number of probe molecules from their carrier liquid and attach the molecules to the walls or to another material positioned within the capillaries.

The capillary bundle can be assembled from a large number of individual, ready-made capillaries. The capillaries can be bundled together, solidified into a single mass or block at their output ends using an adhesive or by fusing the capillary walls at the proximal ends of the capillaries together, and eventually assembled into the print head while the input ends of capillaries are left loose or attached to reservoirs or a plate that dips into a set of reservoirs.

Each capillary can be in fluidic communication to a probe reservoir, which may be a well in a standard microtiter plate. The linkage between the capillary and the reservoir can be made permanent by bonding the capillary to a hole at the bottom of a microplate well. Alternatively, the capillaries can be fixed to a frame which holds the positions of capillary tips in a grid, and which has the same spatial pattern and pitch as a standard microplate. Then, the frame can be locked on to a standard microplate to establish the fluid linkage for each capillary. In this way, the microplate after fabrication can be taken off the arrayer for long-term storage. It is also possible to wash the capillaries after the fabrication of a particular microarray, then install a new set of microplates to make a different microarray.

The output ends of the capillaries may be bonded together into a solid mass. This bonding may be performed using a cement or epoxy that forms a rigid block, or the output ends may be solidified together using a polymer that is somewhat flexible, so that the surface conforms to the substrate to provide better printing in the event that the printing face or facet of the block is not perfectly parallel to the surface of the substrate to be printed. The printing face may optionally be polished to provide a very flat facet, so that the output ends of the capillaries terminate within, for example, 100 μm of each other. In other words, if the printing face is held above and parallel to a plane and separated by a nominal distance z, the difference between the shortest distance that an output end in the facet terminates from the plane and the greatest distance that an output end in the facet terminates from the plane is no more than about 100 μm. In some embodiments, the difference in termination distances is no more than about 50 μm, preferably no more than about 20 μm, and more preferably no more than about 5 μm. The trimmed block can have sufficient rigidity to assure its facet remains parallel to the substrate during printing.

In one embodiment of the invention, the solid mass contains no more than about 10 cm of the lengths of the capillaries (and thus the print head in this embodiment is no more than about 10 cm thick), and the loose or free ends of the capillaries are, for example, from about 1 to about 3 meters in length. Consequently, the ratio of the length of loose capillary to thickness of solid mass is preferably at least about 10 and more preferably at least about 30. The solid mass may be about 2 cm thick or thinner, and in this instance the ratio of length of loose capillary to thickness of solid mass is preferably at least about 50 and more preferably at least about 150. The solid mass may be sufficiently thick such that the print head, alone or in combination with a frame that forms part of the print system, is sufficiently rigid that the solid mass does not deform appreciably under printing conditions, so that a microarray is formed when probes are printed onto a substrate. The loose ends of the capillaries are sufficiently long to be in fluid communication with the reservoirs or with outlet pipes connected to the reservoirs. The loose ends may also be sufficiently long such that the loose portions of the capillaries can accommodate any up-and-down movement of the print head with little stress to the capillaries, so that the capillaries do not crack or break during use.

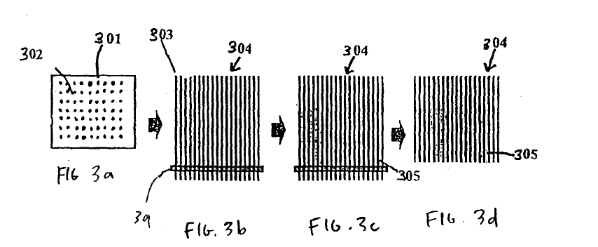

An exemplary guide-plate method for capillary bundle fabrication is illustrated in FIGS. 3A-3D. A guide plate 301 as seen from above in FIG. 3A has an orderly matrix of small holes 302 fabricated through precision drilling. Alternatively, the guide plate can be made of glass and produced by slicing fused capillary array tubing drawn from a larger glass preform as described in U.S. Pat. Nos. 4,010,019 and 5,276,327. The plate can be made of any suitable material, such as, e.g., metal, glass, or plastic, and can also be relatively thin and/or deformable and/or fragile. The hole diameter may be slightly larger than the outer diameter of the capillaries to be used. The guide plate also defines a hole pitch that is defined to be the center-to-center distance of the holes formed in the guide plate for receiving the capillaries. Capillaries 303 are carefully inserted into the holes to form a bundle 304, as illustrated in FIG. 3B. The bundle 304 can be solidified at the section near the guide-plate 301 as shown in FIG. 3C, using epoxy 305, cement or other suitable solidification techniques. Finally, the solidified portion can be cut at a position very close to the guide-plate, to remove the guide plate, as shown in FIG. 3D.

In the above described embodiment, because the holes are positioned in an orderly matrix at the guide-plate and the bundle is cut very close to the guide-plate, the spatial position of each capillary in the fabricated bundle will be in an orderly matrix matching that of the holes in the guide-plate. Also, because the bundle is in one solid piece, it can be polished to achieve a high degree of flatness and at the same time, is mechanically robust for printing. In addition, since the capillaries are in an orderly matrix, the position of each capillary in matrix is known, and therefore the position of the capillary establishes the position of a probe in a microarray printed on a substrate. No ID tagging or other capillary registration procedure is required. A guide plate may be configured in any shape desired. It may be, e.g., a block, a sphere, a plate, or any other shape, so long as the shape has holes, pores, or apertures into which the capillaries may be inserted.

FIG. 4 illustrates a partial cross-section of the capillary bundle in which the capillaries are uniformly spaced into a pattern of rows and columns. The minimum number of capillaries can vary and typically depends upon the number of compounds to be used in a screen. It can be, e.g., more than 100, preferably more than 10³, more preferably more than 10⁴, more preferably more than 10⁵, or more than 10⁶, or more than 10⁷.

Each capillary 402 includes an axial bore 404 having an inner diameter “d”. The inner diameter is selected such that the desired probe-containing fluid is subject to capillary action when inside the capillary 402. Each capillary 402 also includes an outer diameter “D” such that the wall of the capillary 402 has a thickness defined by approximately half of the outer diameter minus half of the inner diameter. The axial bore 404 extends along the length of the capillary 402 from the input end to the output end. The probe-containing fluid is conducted along the axial bore 404 to be printed on the substrate. The outer diameter of each capillary can range from 5 to 500 micrometers, or preferably 30-300 micrometers, or more preferably 40-200 micrometers. The inner diameter of the capillaries can range from, e.g., 1 to 400 micrometers, or preferably 5 to 200 micrometers, or more preferably 10 to 100 micrometers. The spatial capillary pitch P is shown as the center-to-center distance between adjacent capillaries.

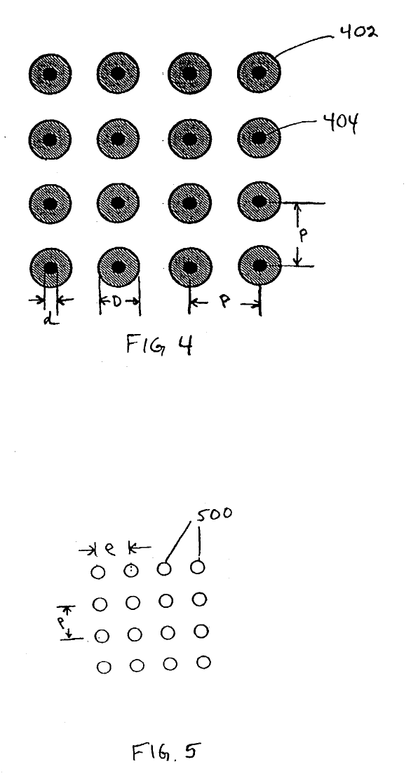

A guide plate can be employed to create a print head having a particular spatial capillary pitch such that the hole pitch on the guide plate is substantially the same as the resulting capillary pitch of the print head. The resulting probe spot that is printed on the substrate by an individual capillary is approximately the same size as the axial bore. For example, if a capillary having a substantially circular cross-section is employed, the resulting printed probe spot will have approximately the same diameter as the axial bore. The spatial probe pitch p is shown as the center-to-center distance between adjacent printed probe spots 500 as shown in FIG. 5. FIG. 5 is representative of a printed portion of a probe array, an un-printed but desired portion of a probe array, or a portion of a patch array formed on a substrate surface for probe binding as described above.

If the capillaries are packed side-by-side such that the outer surface of the capillaries contact each other, the capillary pitch P will be approximately equal to the distance of the outer diameter. If probes are printed using this capillary pitch, then the probe pitch will be equal to the capillary pitch. If printed in this fashion, the density of the resulting printed probe array is limited by the thickness of the capillary wall.

The capillary array in the print head fabricated for differential printing forms a spatial capillary array such that the capillary pitch is equal to a multiple of the desired probe pitch of the array to be printed on the substrate. If the desired probe pitch is p, then the capillary pitch P is equal to an integer multiple of-the desired probe pitch p as expressed by the equation below:

P=Np

In the above equation, P is the capillary pitch and p is the desired probe pitch. Of course, in the variation in which the substrate is formed with functionalized probe patches, the pattern of the patch array corresponds to the general pattern of the capillary array of the print head and the patch pitch is substantially equal to the probe pitch p. N is any integer greater than one, such as, e.g., N=2, 3, 4, 5, or more.

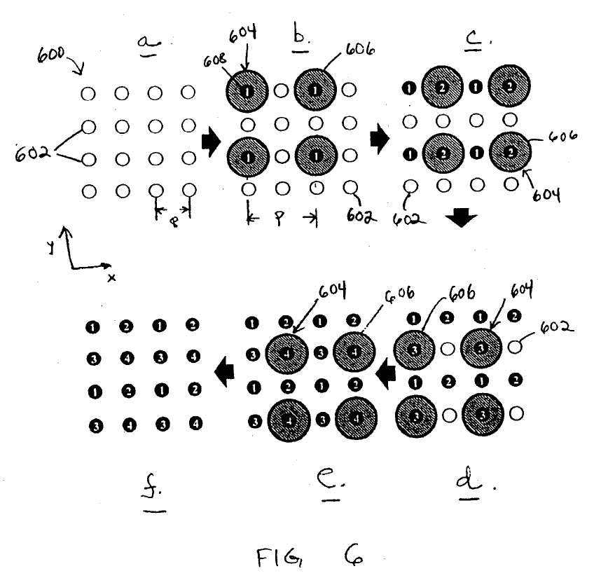

Referring now to FIG. 6, an illustration of a differential printing process in accordance with embodiments of the present invention is provided. In FIG. 6A, there is shown a portion of an array 600 of equally spaced functionalized patches 602 on a substrate surface. The patches 602 form the desired locations for probe spots and are depicted by the smaller open circles. In the variation in which the substrate is not functionalized into patches, the smaller open circles represent the desired probe spots to be printed. The pitch of the patches or desired probe spots is depicted by the letter p. The portion of the array of functionalized patches or of desired probe spots is shown to be a 4×4 array.

In FIG. 6B, a portion of a print head 604 having four capillaries 606 is shown. The cross-sectional footprint of the capillaries 606 is depicted by the large circles. As shown, the capillary pitch P is approximately twice the probe pitch p. The axial bore 608 of each capillary 604 is shown in cross-sectional view. The print head 604 is positioned such that the axial bores 608 of the capillaries 606 are in alignment with a portion of the desired probe spot locations or substrate patch areas 602. A first set of probe spots denoted by numeral “1” is printed as shown in FIG. 6B. The darkened circles identified with the same number are probe printings produced by the same print head or printed in the same step. In embodiments in which the substrate includes probe patches 602, the first set of probe spots can be deposited onto the patch areas 602.

Next, the print head 604 is moved in the x-direction such that the capillaries 606 are positioned above an adjacent set of probe spot locations or probe patches 602 and a second set of probe spots denoted by the numeral “2” is printed, as shown in FIG. 6C. The second set of probe spots is deposited onto the patch areas 602. Next, the print head 604 is moved in the x-direction and y-direction such that the capillaries 606 are positioned above an adjacent set of desired probe spot locations or probe patches 602 and a third set of probe spots denoted by the numeral “3” is printed as shown in FIG. 6D. The third set of probe spots is deposited onto the patch areas 602.

Next, the print head is moved in the x-direction such that the capillaries 606 are positioned above an adjacent set of desired probe spot locations or probe patches 602 if probe patches are employed on the surface and a fourth set of probes denoted by the numeral “4” is printed as shown in FIG. 6E. The resulting printed probe array comprising first, second, third, and fourth sets of prints depositing first, second, third and fourth probe spots, respectively, to form a probe array having a probe pitch p is shown in FIG. 6F.

During the printing process, the same print head and the same capillaries can be employed and moved in the x and y directions to print all of the print sets. However, all four sets of probe spots are not required to be printed by the same print head or same set of capillaries. A different capillary set or sets can be employed for printing one or more probe spot sets. Furthermore, although the physical sequence of prints is shown as 1, 2, 3, 4, any physical sequence is possible. For example, in other variations, the sequence of prints is 1, 2, 4, 3 or 1, 3, 4, 2. To establish a sequence of prints, the print head, translation stage or both may be moved. The movement can be controlled by a processor. Also, the differential printing technique is not limited to capillaries but any fluid dispensing member can be employed with or without an axial bore.

The “chessboard” spatial pattern as shown in FIGS. 6A-6F, for example is a common microarray format. However, differential printing is not limited to this pattern. As mentioned above, the spatial pattern of the capillaries may be determined by that of the holes in the guide plate and on the positioning of the print head during each step. Differential printing can be employed on a honeycomb pattern as well. In a honeycomb pattern, the centers of every three adjacent spots form an equilateral triangle, and six spots surrounding any spot form a hexagon. In addition, the spots align in straight lines globally across the entire microarray. Consequently, the microarray of probes is formed of rows of probe spots, where the probes of every other row (e.g. row n, n+2, n+4, etc. where n=1 or n=2) are also aligned in columns, but an adjacent row is shifted so that a probe of one row lies between two probes of the next row.

As shown in FIGS. 6A-6F, the differential printing process described above can enable print heads with larger capillary size and pitch to print denser microarrays at a high throughtput. The eventual probe density on the substrate surface is N² per unit area of substrate surface. Since the eventual probe density on the substrate surface is N² as much as that on the print head facet, N² number of separate prints have to be conducted in order to produce the microarray. These prints are carried out in a consecutive fashion by either the same print head or different print heads such that 1 to N² number of print heads can be employed.

High density microarray production is possible using the differential printing process. For example, a typical useable area on a standard microscope slide is 18 mm×60 mm. Print heads with a pitch of 120 μm can print a maximum of 75,000 spots without the differential printing technique presented above. With double differential printing, the probe pitch can be reduced to 60 μm yielding a total of 300,000 probes. This invention significantly reduces the chance of probe cross-talk during the printing and increases production yield. The same technique can be used in other areas that require high density fluid delivery which include protein chips, compound chips, high throughput screen chips, etc.

In the system shown in FIG. 2, multiple microarray substrates are carried on a translation stage, which moves in at least direction in a stepping fashion to align a blank substrate under the print head. The translation stage can be a rotation stage or a conveyor belt based system equipped with substrate loading and unloading stations. In this way, blank substrates can be fed to a print position beneath the print head in a continuous fashion. The print head can deposit at least a first set of probes by moving only a very short distance (<1 mm) along any one axis (up and down in the z axis). Or the print head may not have to move at all if electric or magnetic induced deposition methods are used. As a result, microarray manufacturing can be carried out in a continuous fashion at a very high throughput.

The arrayer system further includes a fluid-delivery sub-system, probe deposition system, and inspection system. These and other basic elements and other methods are discussed in PCT Publication No. WO 01/62377 published on Aug. 30, 2001, the entire contents of which is incorporated herein by reference. In general, the fluid delivery sub-system transports probe fluid from the reservoir to the print head through its respective capillary. The fluid delivery sub-system also ensures that the flow rate is constant in each capillary and uniform across the print head. Several methods can be employed to drive the probe fluid from its reservoir into the capillary and towards the print head. These methods include use of differential air pressure, gravity, electric field, and vacuum can be used alone or in combination.

The arrayer system also includes a probe deposition subsystem that ensures that a constant and uniform volume of probe fluids are deposited onto the substrate and there are minimal or no missing or overlapped spots on the microarray. Probes can be deposited on the microarray substrate by mechanically tapping the print head on the substrate in which the constant flow of probe solution in the capillary produces a micro sphere of fluid at the facet of each capillary. When the print head is tapped on the substrate, the droplet bonds to the substrate due to surface tension. Furthermore, electrostatic printing methods can be employed to print the array. Also, probes may be immobilized on printing beads and a colloidal suspension formed, and the suspension can be deposited through the capillaries and onto the substrate to deposit the beads onto the substrate. Electromagnetic printing can also be employed in which probe molecules are attached to ferrofluids to form ferrofluid particles and deposited on the substrate. Yet another method is vacuum printing in which the output ends of the capillaries are placed under relative vacuum in order to draw probe-containing fluid through the capillaries.

In one variation, the arrayer is configured to deposit more than one layer of the same or different materials onto the same desired spot location or patch. In such a variation, the printing system includes at least one probe reservoir and a substrate configured to receive a probe array having a probe pitch p. A plurality of fluid dispensing members, each having a proximal and a distal end, are also provided. Each fluid dispensing member is in fluid communication with at least one probe reservoir. The proximal ends are secured to be substantially coplanar in an array in a facet of the print head such that the fluid dispensing members have a pitch P. In one variation, the fluid dispensing members are capillaries.

The print head is configured to print an array of first material. The first material is then allowed to dry in one variation before a second material is printed. Then, a second material is printed onto the same array locations as the printed array of first material. Typically, a second print head is employed to deposit the second array of second material. This second print head has the same spatial pitch and pattern as the first printed array. This second print head is aligned with the first deposited array of first material before depositing the second material onto the location of the first array. The second material deposited in the second print is then allowed to dry in one variation before a subsequent material is deposited. This process can be repeated to deposit multiple materials onto into a single array, stacking layer upon layer. Any one of the series of prints may be performed using differential printing techniques described above, however, the invention is not so limited and non-differential printing can be employed to lay down any one layer of material. The deposited materials can each be any mixture of different biochemical materials. In one example, the first material is deposited into an array to functionalize the substrate surface. The second material that is deposited onto the same array location is a probe-containing agent and a third material that is deposited is a marker or indicator for providing a signal. A solid phase assay of multiple materials is thereby created by adding different reagents to the same array locations in separate prints.

Capillary Registration

In accordance with other aspects of the present invention, methods for registering the identity of specific capillaries in a capillary bundle are provided. The association between the proximal and distal ends of each capillary in the capillary bundle should be identified and maintained so that the identity of each reagent delivered to the proximal end can be established. However, such an association can be easily lost during the bundling process when the number of capillaries in the bundle becomes very large. The above-listed patent applications describe methods of re-establishing this association after the bundling process has been completed. Because the proximal end has been solidified after bundling, the relative position of each capillary in the facet of the proximal end is fixed and can be used to register its identity. This process of re-establishing capillary association after bundling can be referred to as “ID tagging”. A number of ID tagging techniques are described in the above-listed applications. In accordance with embodiments of the present invention, additional ID tagging methods are provided below.

In accordance with embodiments of the present invention, an ID tagging method involves producing a set of fluid ID tags. Each fluid ID tag is individually identifiable. The ID tags are loaded into reagent reservoirs and transported from the distal end of the capillary to the proximal end. By identifying the identity of the ID tag, the association between the capillary in the proximal and distal end can be re-established.

In one embodiment, the ID tags are fluids of different colors. The proximal end is used to imprint on a material to form a spot array. The color of each spot is analyzed and the identity of associated capillary is identified. The colored fluid can be, for example, a mixture of different dyes and the instrument for color analysis can be a microarray scanner with two or four color capability. The spot array may be imprinted on a white material so as to enable easy identification of the fluid colors.

In another embodiment, each ID tag is a unique mixture of different oligonucleotides. After loading the tags into capillaries, the proximal end of the bundle is used to print on a microarray substrate to produce a microarray. The specific oligonucleotide mixture can be identified by hybridization with the compliments of the oligonucleotides in the mixture.

In one embodiment utilizing the oligonucleotide tags, each tag (or mixture) is comprised of M number of oligonucleotides, which are selected so as to be of high specificity and minimum cross-hybridization. Among these M oligonucleotides, one oligonucleotide, Oᵣ, will be used as the “reference oligonucleotide”, with the others being referred to as “coding oligonucleotides”. In each tag, the reference oligonucleotide always has the same relative concentration, while the relative concentrations of coding oligonucleotides may vary. The concentration combination of coding oligonucleotides generates a unique “code” that identifies a particular mixture. Table 1 illustrates an exemplary coding system in which 4 coding oligonucleotides and 2 different concentrations (1 and 10 μM) each produce 2⁴=16 different tags.

TABLE 1

Coding Oligonucleotide

| | Concentration (μM) | | | | Ref | Total |
|---|---|---|---|---|---|---|
| Tag ID | O1 | O2 | O3 | O4 | Oᵣ | (μM) |
| 1 | 1 | 1 | 1 | 1 | 1 | 5 |
| 2 | 1 | 1 | 1 | 10 | 1 | 14 |
| 3 | 1 | 1 | 10 | 1 | 1 | 14 |
| 4 | 1 | 1 | 10 | 10 | 1 | 23 |
| 5 | 1 | 10 | 1 | 1 | 1 | 14 |
| 6 | 1 | 10 | 1 | 10 | 1 | 23 |
| 7 | 1 | 10 | 10 | 1 | 1 | 23 |
| 8 | 1 | 10 | 10 | 10 | 1 | 32 |
| 9 | 10 | 1 | 1 | 1 | 1 | 14 |
| 10 | 10 | 1 | 1 | 10 | 1 | 23 |
| 11 | 10 | 1 | 10 | 1 | 1 | 23 |
| 12 | 10 | 1 | 10 | 10 | 1 | 32 |
| 13 | 10 | 10 | 1 | 1 | 1 | 23 |
| 14 | 10 | 10 | 1 | 10 | 1 | 32 |
| 15 | 10 | 10 | 10 | 1 | 1 | 32 |
| 16 | 10 | 10 | 10 | 10 | 1 | 41 |

As described above, the ID tags are loaded into the bundle with one tag per capillary. The proximal end is used as a print head to imprint a microarray of oligonucleotide spots on a substrate. A minimum of M microarrays are produced. These M microarrays will be hybridized with the compliments of M oligonucleotides in the mixture in M separate synthetic hybridizations. Only the compliment of one coding oligonucleotide and the compliment of the reference oligonucleotide will be used in any particular hybridization. The compliment of the coding oligonucleotide and that of the reference oligonucleotide should be labeled with different dyes. The target oligonucleotide should be abundant in the hybridization. After hybridization, the microarray can be read in a microarray scanner. The relative concentration between the reference oligonucleotide and one of the coding oligonucleotides can be obtained through the relative strength of the fluorescence signal. The relative concentration of all coding oligonucleotides can be obtained by repeating the hybridization process M number of times, each time with a target complimentary to a different one of the M oligonucleotides. Then, the identity of the tags at each spot can be obtained through the unique distribution of relative concentrations among coding oligonucleotides. And the identity of the capillary at the corresponding positions can be obtained.

As shown in Table 1, the total oligonucleotide concentration of each tag is different in the exemplary design. This may introduce a bias in hybridization efficiency. To solve this problem, an additional “compensation oligonucleotide” can be introduced into each tag which makes up the total concentration to a set level for all tags, as illustrated in Table 2.

TABLE 2

| | | | |
|---|---|---|---|
| Coding Oligonucleotide | | | |
| concentration (μM) | Ref | Comp. | Total |

| Tag ID | O1 | O2 | O3 | O4 | Or | Oc | (μM) |
|---|---|---|---|---|---|---|---|
| 1 | 1 | 1 | 1 | 1 | 1 | 36 | 41 |
| 2 | 1 | 1 | 1 | 10 | 1 | 27 | 41 |
| 3 | 1 | 1 | 10 | 1 | 1 | 27 | 41 |
| 4 | 1 | 1 | 10 | 10 | 1 | 18 | 41 |
| 5 | 1 | 10 | 1 | 1 | 1 | 27 | 41 |
| 6 | 1 | 10 | 1 | 10 | 1 | 18 | 41 |
| 7 | 1 | 10 | 10 | 1 | 1 | 18 | 41 |
| 8 | 1 | 10 | 10 | 10 | 1 | 9 | 41 |
| 9 | 10 | 1 | 1 | 1 | 1 | 27 | 41 |
| 10 | 10 | 1 | 1 | 10 | 1 | 18 | 41 |
| 11 | 10 | 1 | 10 | 1 | 1 | 18 | 41 |
| 12 | 10 | 1 | 10 | 10 | 1 | 9 | 41 |
| 13 | 10 | 10 | 1 | 1 | 1 | 18 | 41 |
| 14 | 10 | 10 | 1 | 10 | 1 | 9 | 41 |
| 15 | 10 | 10 | 10 | 1 | 1 | 9 | 41 |
| 16 | 10 | 10 | 10 | 10 | 1 | 0 | 41 |

The hybridization efficiencies of the reference and coding oligonucleotides may be different due to different sequences. This bias can be quantified through experiments and mathematically compensated in the final calculation. In general, C coding oligonucleotides and K different concentrations generate K^{C} different ID tags. For example, 6 coding oligonucleotides in 5 concentrations produce 15,625 unique tags, which is sufficient to ID tag a bundle of 10,000 capillaries.

While the invention has been described in terms of particular embodiments and illustrative figures, those of ordinary skill in the art will recognize that the invention is not limited to the embodiments or figures described. For example, it was noted above that in some embodiments, the locations of the individual capillaries of print heads formed in an orderly matrix are known, thereby obviating the need for an ID tagging registration. However, in other embodiments, the capillary registration methods described herein could be applied to any type of capillary bundle, regardless of the organization of capillaries and the manufacturing process. For example, the ID tagging process may be used to confirm the expected associations of proximal and distal ends of each capillary.

In addition, the methods and steps described above indicate certain events occurring in a certain order. Those of ordinary skill in the art will recognize that the ordering of certain steps may be modified, and that such modifications are in accordance with the various embodiments of the invention. Additionally, certain of the steps may be performed concurrently in a parallel process when possible, as well as performed sequentially as described above.

Therefore, it should be understood that the invention can be practiced with modification and alteration within the spirit and scope of the appended claims. The description is thus to be regarded as illustrative instead of limiting on the invention.

1. A method for printing a microarray comprising:
providing a substrate having a substrate surface;
providing at least one probe reservoir;
providing at least one capillary bundle comprising a plurality of individual capillaries; each of the capillaries having an input end and an output end; wherein the output ends of the individual capillaries are secured in a print head such that the output ends of the capillaries are substantially coplanar in an array in a facet of the print head such that the capillaries have a capillary pitch P;
placing the input ends of the individual capillaries in fluid communication with at least one probe reservoir;
transporting probe from at least one probe reservoir to the output ends of the capillaries; and
printing an array of probes on the substrate such that the printed probes have a probe pitch of approximately PIN; wherein N is an integer greater than one.

2. The method of claim 1 wherein printing an array of probes further includes the step of printing the array of probes in N² number of separate prints.

3. A method for printing a microarray, comprising:
providing a substrate for receiving a probe array having a probe pitch p;
providing at least one probe reservoir;
providing at least one capillary bundle comprising a plurality of individual capillaries; each of the capillaries having an input end and an output end; wherein the output ends of the individual capillaries are secured in a print head such that the output ends of the capillaries are substantially coplanar in an array in a facet of the print head such that the capillaries have a capillary pitch P; wherein the capillary pitch P is an integer multiple of the probe pitch p; wherein the integer is greater than one;
placing the input ends of the individual capillaries in fluid communication with at least one probe reservoir;
transporting probe from the at least one probe reservoir to the output ends of the capillaries; and
printing N2 number of prints to deposit N2 sets of probes onto the substrate to form a probe array having a probe pitch p.

4. A method for printing a microarray comprising:
providing a substrate having a substrate surface;
providing at least one reservoir;
providing at least one print head comprising a plurality of fluid dispensing members having a distal end and a proximal end; each fluid dispensing member being in fluid communication with at least one reservoir; wherein the proximal ends of the individual fluid dispensing members are secured such that the proximal ends of the fluid dispensing members are substantially coplanar in an array in a facet of the print head;
printing a first array of first material onto the substrate; and
printing at least a second array of at least second material onto the first array.

5. A method for printing a microarray using at least one capillary bundle comprising a plurality of individual capillaries, each of the capillaries having an input end and an output end, wherein the output ends of the individual capillaries are secured in a print head, said method comprising:
transporting probe from at least one probe reservoir to the output ends of the capillaries;
printing a first array of probes on a substrate; and
printing a second array of probes on the substrate, said second array of probes overlapping and offset from the first array of probes such that at least some of the probes in the second array of probes are located between the probes in the first array of probes.

6. A method of associating proximal and distal ends of a plurality of capillaries in a capillary bundle, said method comprising:
loading a plurality of fluids into the distal ends of the plurality of capillaries, each capillary having a unique fluid being loaded therein;
transporting the plurality of fluids from the distal ends of the plurality of capillaries to the proximal ends;
printing the plurality of fluids from the proximal ends of the plurality of capillaries onto a substrate to form an array of spots, each spot corresponding to one of the plurality of fluids; and
registering one of the capillaries by identifying the fluid forming one of the spots in the array of spots, matching the identified fluid with one of the plurality of fluids loaded into the distal ends of the capillaries, and correlating the location of the spot with the capillary loaded with the matched fluid.

7. The method of claim 6, wherein said loading a plurality of fluids into the distal ends of the plurality of capillaries comprises:
loading a plurality of fluids into the distal ends of the plurality of capillaries, each fluid including a unique combination of one or more oligonucleotides, each of the one or more oligonucleotides having a known sequence.

8. The method of claim 7, wherein said identifying the fluid forming one of the spots in the array of spots comprises:
hybridizing the array of spots with a target solution including targets complimentary to one of the oligonucleotides in the unique combination of one or more oligonucleotides.

9. The method of claim 7, wherein said loading the plurality of fluids into the distal ends of the plurality of capillaries comprises:
loading a plurality of fluids into the distal ends of the plurality of capillaries, each fluid including: (1) a reference oligonucleotide having a known sequence; and (2) the unique combination of one or more oligonucleotides.
